(19) United States
(12) Patent Application Publication
(10) Pub. No.: US 2005/0181497 A1
(43) Pub. Date: Aug. 18, 2005

(54) Solid substrate used for sensors

(75) Inventors: Yukou Saito, Kanagawa (JP); Hirohiko Tsuzuki, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/003,445

(22) Filed: Dec. 6, 2004

(30) Foreign Application Priority Data
Dec. 4, 2003 (JP) 405704/2003
Jan. 8, 2004 (JP) 002926/2004
Feb. 3, 2004 (JP) 026607/2004

(51) Int. Cl. C12M 1/34

(52) U.S. Cl. 435/287.1

(57) ABSTRACT

It is an object of the present invention to provide a solid substrate used for sensors that suppresses nonspecific adsorption and that is able to immobilize a physiologically active substance. The present invention provides A solid substrate used for sensors, wherein two or more different hydrophobic polymer layers are laminated on the solid substrate, and among the above hydrophobic polymer layers, the surface of a layer, which is farthest from the solid substrate, is modified.

### TECHNICAL FIELD

The present invention relates to a solid substrate used for sensors, which prevents nonspecific adsorption. More specifically, the present invention relates to a solid substrate used for sensors that is able to immobilize a physiologically active substance on the outermost surface thereof and that prevents nonspecific adsorption. The present invention relates to a method for producing a solid substrate. More particularly, the present invention relates to a method for producing a solid substrate used for sensors.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No 2815120). Moreover, a measurement chip comprising a metal film and a plasma-polymerized film formed on the metal film has been reported (Japanese Patent Laid-Open No. 9-264843).

When a specific binding reaction between a physiologically active substance and a test substance is measured, the test substance is not necessarily comprised of a single component. There may also be a case where a test substance is required to be measured in a heterogeneous system such as a cell extract. In such a case, if contaminants such as various proteins or lipids are adsorbed on the detection surface nonspecifically, measurement/detection sensitivity is significantly reduced. The fact that nonspecific adsorption is highly likely to occur on the above detection surface has been problematic.

In order to solve such problems, several methods have been studied. For example, a method of immobilizing a hydrophilic hydrogel on a metal surface via a linker, so as to repress physical adsorption, has been used (Japanese Patent No. 2815120, U.S. Pat. No. 5,436,161, and Japanese Patent Laid-Open No. 8-193948). However, nonspecific adsorption has not been sufficiently controlled by this method.

The aforementioned nonspecific adsorption can also be suppressed by forming on the surface of a sensor substrate a thin hydrophobic polymer film, which does not react with any organism-related substance. Examples of conventional methods of forming a thin hydrophobic polymer film on a sensor substrate may include spin coating, air knife coating, cast coating, and spray coating. In such methods, a polymer solution is applied on a substrate, and then a solvent is removed by drying. However, such methods are problematic in that pinholes or an uneven thickness are likely to be generated on a thin film when it is dried. In addition, the surface of the above substrate is required to be planar to prevent uneven application of the solution. A metal film and a plasma-polymerized film formed on the metal film have been reported. However, since a monomer material is applied by coating, the same above problems still remain (Japanese Patent Laid-Open No. 9-264843). A method involving evaporating a monomer material onto a substrate and then polymerizing it on the substrate has also been reported, but it is problematic in that the types of monomers that can be used are limited (Japanese Patent Laid-Open No. 2003-212974).

It is reported that a laminated film of a combination of certain hydrophobic polymers can be formed on a QCM substrate by an adsorption method (Langmuir. 2000, 17, 5513-5519). However, a sensing method for suppressing nonspecific adsorption using this laminated film and measuring the binding of a physiologically active substance and a test substance has not yet been proposed.

### DISCLOSURE OF THE INVENTION

It is an object of the present invention to solve the aforementioned problems of the prior art techniques. In other words, it is an object of the present invention to provide a solid substrate used for sensors that suppresses nonspecific adsorption and that is able to immobilize a physiologically active substance. Further, it is an object of the present invention to provide a method for producing the solid substrate that suppresses nonspecific adsorption, particularly a method for producing the solid substrate used for sensors that controls nonspecific adsorption which is used for, for example, a surface plasmon resonance analysis.

As a result of intensive studies directed towards achieving the aforementioned object, the present inventors have found that a solid substrate used for sensors, which is produced by alternatively laminating two or more different hydrophobic polymer layers on a solid substrate, and modifying a layer that is farthest from the solid substrate, is used, so as to immobilize a physiologically active substance on the solid substrate, while nonspecific adsorption is suppressed. Further, the present inventors have found that a solid substrate that allows various hydrophobic polymers to adsorb on the surface thereof and that suppresses nonspecific adsorption, can be produced by a surface-forming method, which comprises steps of allowing the solid substrate to come into contact with a hydrophobic polymer solution and then allowing it to come into contact with a mixed solution comprising two or more organic solvents, which does not contain the above polymer. The present invention has been completed based on these findings.

That is to say, the first aspect of the present invention provides a solid substrate used for sensors, wherein two or more different hydrophobic polymer layers are laminated on the solid substrate, and among the above hydrophobic polymer layers, the surface of a layer, which is farthest from the solid substrate, is modified.

The surface-modified hydrophobic polymer layer preferably has a functional group capable of generating a covalent bond.

The solid substrate preferably has one or more holes or projections on the surface thereof. The projected area of the aforementioned hole or projection observed from the top of the substrate is between 0.001 mm² and 10,000 mm². The depth or height of the aforementioned hole or projection is between 100 nm and 10 cm.

Preferably, a metal film exists between the solid substrate and the hydrophobic polymer layer.

The metal film preferably consists of a free electron metal selected from the group consisting of gold, silver, copper, platinum, and aluminum.

The surface-modified hydrophobic polymer layer preferably has a functional group capable of immobilizing a physiologically active substance.

The functional group capable of immobilizing a physiologically active substance is preferably —OH, —SH, —COOH, —NR¹R² (wherein R¹ and R² each independently represents a hydrogen atom or lower alkyl group), —CHO, —NR³NR¹R² (wherein each of R¹, R², and R³ independently represents a hydrogen atom or lower alkyl group), —NCO, —NCS, an epoxy group, or a vinyl group.

The solid substrate used for sensors of the present invention is preferably used in non-electrochemical detection, and more preferably in surface plasmon resonance analysis.

In another aspect, the present invention provides a method for producing the solid substrate used for sensors of the present invention, which comprises steps of allowing two or more types of hydrophobic polymer solutions to come into contact with a solid substrate in turns, and modifying the surface of the obtained solid substrate.

In a further aspect, the present invention provides a method for producing a solid substrate used for sensors, to the surface of which a physiologically active substance binds; wherein the method comprises a step of allowing the physiologically active substance to come into contact with the surface of the solid substrate used for sensors of the present invention, so as to immobilize the substance thereon.

In a further aspect, the present invention provides the aforementioned solid substrate used for sensors of the present invention, to the surface of which a physiologically active substance binds.

In a further aspect, the present invention provides a method for detecting or measuring a substance interacting with a physiologically active substance, which comprises steps of allowing the physiologically active substance to come into contact with the surface of the solid substrate used for sensors of the present invention, so as to immobilize the substance thereon, and allowing the obtained solid substrate used for sensors, to the surface of which the physiologically active substance binds, to come into contact with a test substance.

The second aspect of the present invention provides a method for producing a solid substrate used for sensors which comprises steps of allowing a solid substrate to come into contact with a hydrophobic polymer solution and then allowing it come into contact with a mixed solution comprising two or more organic solvents, which does not contain the above polymer.

There is preferably provided a method for producing a solid substrate used for sensors, which further comprises a step of modifying the surface of the obtained solid substrate.

The above mixed solution comprising two or more organic solvents, which does not contain the polymer, preferably comprises a good solvent and a poor solvent for the above polymer.

The mixed solution comprising two or more organic solvents, which does not contain the above polymer, is preferably used at a liquid temperature that is 1° C. to 50° C. higher than the lower limit liquid temperature at which no hydrophobic polymer deposits are generated when the concentration of the above mixed solution is adjusted to the same concentration as that of the above hydrophobic polymer solution containing hydrophobic polymers.

More preferably, a solvent contained in the hydrophobic polymer solution is identical to a solvent contained in the solution, which does not contain the polymer. Preferably, the above surface modification involves introduction of a functional group capable of generating a covalent bond. Preferably, the solid substrate, which is allowed to come into contact with the hydrophobic polymer solution, has a metal surface or is coated with a metal film. Preferably, the aforementioned solid substrate used for sensors is used in surface plasmon resonance analysis.

In another aspect, the present invention provides a method for producing a solid substrate used for sensors, to the surface of which a physiologically active substance binds, wherein the above method comprises steps of producing a solid substrate used for sensors by the aforementioned method of the present invention, and allowing a physiologically active substance to come into contact with the surface of the obtained solid substrate used for sensors, so as to immobilize the substance thereon.

In a further aspect, the present invention provides a method for detecting or measuring a substance interacting with a physiologically active substance, wherein the above method comprises steps of producing a solid substrate used for sensors by the aforementioned method of the present invention, allowing the physiologically active substance to come into contact with the surface of the obtained solid substrate used for sensors, so as to immobilize the substance thereon, and allowing the obtained solid substrate used for sensors, to the surface of which the physiologically active substance binds, to come into contact with a test substance.

### BEST MODE FOR CARRYING OUT THE INVENTION

The embodiments of the present invention will be described below.

The solid substrate used for sensors of the present invention is characterized in that two or more different hydrophobic polymer layers are laminated on the solid substrate, and that among the above hydrophobic polymer layers, the surface of a layer, which is farthest from the solid substrate, is modified.

The method of the present invention for producing a solid substrate used for sensors is characterized in that a solid substrate is allowed to come into contact with a hydrophobic polymer solution and then allowed to come into contact with a mixed solution comprising two or more organic solvents, which does not contain the above polymer, so as to form a surface thereof.

In the solid substrate used for sensors according to the present invention, two or more different hydrophobic polymers are used. The hydrophobic polymer used in the present invention is substantially insoluble in water. Specifically, the solubility of the hydrophobic polymer in water is less than 0.1%. The hydrophobic polymer used in the present invention preferably comprises a monomer that represents 30% to 100% by weight based on the weight of such polymer. The solubility in water of the aforementioned monomer at 25° C. is between 0% by weight and 20% by weight.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

The type of solvent for dissolving the polymer which is used in the present invention is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

The thickness of the hydrophobic polymer layer is not particularly limited. The total thickness of all the laminated polymer layers is preferably between 1 angstrom and 5,000 angstroms, and particularly preferably between 10 angstroms and 3,000 angstroms.

A substrate is coated with the above-described high polymer according to common methods. Examples of such a coating method may include spin coating, air knife coating, bar coating, blade coating, slide coating, curtain coating, spray method, evaporation method, cast method, and dip method.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. It is preferred that the hydrophobic polymer is dissolved completely. The temperature of the solution is not particularly limited, so long as the state of the solution allows a part of the hydrophobic polymer to be dissolved. The temperature is preferably higher than the temperature of the solution at which a hydrophobic polymer generates precipitates. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ was calculated by the following formula. As the cohesive energy (Ecoh) of each functional group and the mol volume (V), those defined by Fedors were used (R. F. Fedors. Polym. Eng. Sci., 14(2), P147, P472 (1974)).
δ=(ΣEcoh/ΣV)^{1/2}

The SP values of the hydrophobic polymers and the solvents used in the Examples are shown below;
Solvent: 2-phenoxyethanol: 25.3 against polymethylmethacrylate-polystyrene copolymer (1:1): 21.0
Solvent: acetonitrile: 22.9 against polymethylmethacrylate: 20.3
Solvent: toluene: 18.7 against polystyrene: 21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

In the method for producing a solid substrate for sensors according to the present invention, a solid substrate is allowed to come into contact with the aforementioned hydrophobic polymer solution, and it is then allowed to come into contact with a mixed solution comprising two or more organic solvents, which does not contain the above polymer. The term a “mixed solution comprising two or more organic solvents, which does not contain the above polymer” is used in the present invention to mean organic solvents containing no polymers. It is preferably a mixed solution comprising a good solvent and a poor solvent for polymers. The liquid temperature of the solvents containing no polymers is preferably 1° C. to 50° C. higher than the lower limit liquid temperature at which no polymer agglutinates are generated. Moreover, a solvent contained in the hydrophobic polymer solution is preferably identical to a solvent contained in the mixed solution comprising two or more organic solvents, which contains the polymer, in terms of composition.

The term a “good solvent” is used in the present invention to mean a solvent in which the solubility of a polymer is 0.1% or more. The term a “poor solvent” is used in the present invention to mean a solvent in which substantially no polymers are dissolved. For example, when polymethyl methacrylate is used as a polymer, examples of a good solvent used herein may include acetone, acetonitrile, benzene, 2-butanone, tetrahydrofuran, acetic acid, ethyl acetate, chloroform, chlorobenzene, methylene chloride, cyclohexanone, dioxane, and 2-ethoxyethanol. Examples of a poor solvent used herein may include cyclohexane, dimethyl ether, ethylene glycol, formamide, hexane, methanol, ethanol, carbon tetrachloride, cresol, and naphthalene. Examples of a good solvent and a poor solvent for hydrophobic polymers may include those described in “Polymer Handbook Fourth Edition” Chapter 4, pp. 497 to 545, edited by J. Brandrup, E. H. Immergut, and E. A. Grulke, John Wiley & Sons (1999).

In the present invention, the liquid temperature of the mixed solution comprising two or more organic solvents containing no polymers is not particularly limited. However, it is preferably a liquid temperature at which no hydrophobic polymer deposits are generated when the concentration of the above mixed solution is adjusted to the same concentration as that of the above hydrophobic polymer solution containing hydrophobic polymers used also in the present invention. Specifically, it is preferably a liquid temperature 1° C. or more higher than the lower limit liquid temperature at which no polymer deposits are generated. Further, for the purpose of increasing the liquid temperature to prevent the generated hydrophobic polymers from leaving the solid substrate, the liquid temperature is preferably 50° C. or less higher than the aforementioned lower limit liquid temperature.

The period of time necessary for allowing the substrate to come into contact with the mixed solution comprising two or more organic solvents containing no polymers is not particularly limited. It is preferably between 1 second and 24 hours, and more preferably between 3 seconds and 1 hour. The liquid temperature is not particularly limited, as long as the solvent is in a liquid state. It is preferably between −20° C. and 100° C. It may also be possible for the liquid temperature to fluctuate during the time when the substrate is allowed to come into contact with the solvent. In the case of using a solvent that is hardly volatilized, after the substrate has been allowed to come into contact with the solvent, the solvent may be substituted with a volatile solvent, so that both solvents are dissolved in each other, and so that the above solvent can be eliminated.

In the present invention, after a hydrophobic polymer solution is allowed to come into contact with a solid substrate, the surface of the obtained solid substrate is modified. Such a surface modification method can be selected, as appropriate, from chemical treatments using chemical agents, coupling agents, surfactants, or surface evaporation, and physical treatments using heating, ultraviolet rays, radioactive rays, plasma, or ions.

It is preferable that a functional group capable of generating a covalent bond as a result of surface modification be introduced into the surface-modified layer in the present invention. Preferred functional group includes —OH, —SH, —COOH, —NR¹R² (wherein each of R¹ and R² independently represents a hydrogen atom or lower alkyl group), —CHO, —NR³NR¹R² (wherein each of R¹, R² and W³ independently represents a hydrogen atom or lower alkyl group), —NCO, —NCS, an epoxy group, or a vinyl group. The number of carbon atoms contained in the lower alkyl group is not particularly limited herein. However, it is generally about C1 to C10, and preferably C1 to C6.

In order to introduce these functional groups into the surface, a method is applied that involves applying a hydrophobic polymer containing a precursor of such a functional group on a metal surface or metal film, and then generating the functional group from the precursor located on the outermost surface by chemical treatment. For example, polymethyl methacrylate, a hydrophobic polymer containing —COOCH₃ group, is applied on a metal film, and then the surface comes into contact with an NaOH aqueous solution (1N) at 40° C. for 16 hours, so that a —COOH group is generated on the outermost surface. In addition, when a polystyrene coating layer is subjected to a UV/ozone treatment for example, a —COOH group and a —OH group are generated on the outermost surface thereof.

The term “solid substrate” is interpreted in the broadest sense in the present invention. It means a base for supporting a material having functions. It does not only include solid bases, but also includes those consisting of flexible materials, such as a film or sheet.

The solid substrate of the present invention has one or more holes or projections on the surface thereof. It is preferable that the projected area of the aforementioned hole or projection observed from the top of the substrate be between 0.001 mm² and 10,000 mm², and that the depth or height thereof be between 100 nm and 10 cm.

The position of the hole or projection may be either a position where a test substance is not placed, or a position where a test substance is placed. In addition, the hole or projection can be formed at any given position. A projection may be formed at the bottom of a hole, or a hole may be formed at the top of a projection. For example, a projection is used as an aligner mark or spacer, so that the position between a detection surface and a measurement device can precisely be designed. Furthermore, for example, when a test substance is introduced from such a projection or hole portion, a solution is added dropwise to individual projection or hole portions, and a reaction such as a chemical reaction or binding reaction is individually carried out in the solution, thereby performing detection.

It is preferred that the solid substrate used in the present invention is obtained by coating a metal surface or a metal film with a hydrophobic polymer. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 1 angstrom and 5,000 angstroms, and particularly preferably between 10 angstroms and 2,000 angstroms. If the thickness exceeds 5,000 angstroms, the surface plasmon phenomenon of a medium cannot be sufficiently detected. Moreover, when an interstitial layer consisting of chrome or the like is provided, the thickness of the interstitial layer is preferably between 1 angstrom and 100 angstroms.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and having excellent workability are preferably used.

The solid substrate of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

A physiologically active substance is covalently bound to the above-obtained substrate for sensor via the above functional group, so that the physiologically active substance can be immobilized on the metal surface or metal film.

A physiologically active substance immobilized on the substrate for sensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against 0 antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

A biosensor to which a physiologically active substance is immobilized as described above can be used to detect and/or measure a substance which interacts with the physiologically active substance.

In the present invention, it is preferable to detect and/or measure an interaction between a physiologically active substance immobilized on the solid substrate for sensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example A-1

### it/st-PMMA/COOH Surface Block

(1) Preparation of Isotactic-Polymethyl Methacrylate Solution (0.2% it-PMMA)

0.2 g of isotactic-polymethyl methacrylate (number average molecular weight: 23,000; hereinafter referred to as it-PMMA) was dissolved in 100 ml of acetonitrile to prepare 0.2% it-PMMA.

(2) Preparation of Syndiotactic-Polymethyl Methacrylate Solution (0.2% st-PMMA)

0.2 g of syndiotactic-polymethyl methacrylate (number average molecular weight: 23,000; hereinafter referred to as st-PMMA) was dissolved in 100 ml of acetonitrile to prepare 0.2% st-PMMA.

(3) Production of Gold Block

Gold was evaporated onto the dielectric block shown in FIG. 23 of Japanese Patent Laid-Open (Kokai) No. 2001-330560, such that the thickness of a gold film became 500 angstroms, so as to obtain a gold block.

(4) Production of it/st-PMMA Alternatively Laminated Block

The gold block was treated with a Model-208 UV-ozone cleaning system (TECHNOVISION INC.) for 30 minutes. Thereafter, 0.2% it-PMMA was added dropwise to the surface coated with gold via evaporation, and it was then left at rest for 15 minutes. Subsequently, the above block was immersed in 50 ml of acetonitrile 5 times each for 1 minute, so that 0.2% it-PMMA attached to the surface coated with gold via evaporation was substituted with acetonitrile. After completion of the substitution, acetonitrile attached to the surface of the block was removed by nitrogen blowing. Subsequently, 0.2% st-PMMA was added dropwise to the surface of the block upon which gold has been deposited, and it was then left at rest for 15 minutes. Subsequently, the above block was immersed in 50 ml of acetonitrile 5 times each for 1 minute, so that 0.2% st-PMMA attached to the surface coated with gold via evaporation was substituted with acetonitrile. After completion of the substitution, acetonitrile attached to the surface of the block was removed by nitrogen blowing. These operations were repeated 4 times, so as to form a hydrophobic polymer layer consisting of 4 it-PMMA layers and 4 st-PMMA layers that were alternatively laminated. The thickness of the film was measured by the ellipsometry method (In-Situ Ellipsometer MAUS-101; manufactured by Five Lab). As a result, the thickness of the it/st-PMMA alternatively laminated film was found to be 40 angstroms. This sample was called an it/st-PMMA alternatively laminated block.

(5) it/st-PMMA/COOH Surface Block

The it/st-PMMA alternatively laminated block was immersed in an NaOH aqueous solution (1 N) at 40° C. for 16 hours. Thereafter, the block was washed with water 3 times, and the water was then removed by nitrogen blowing. The thickness of an it/st-PMMA/COOH film was measured by the ellipsometry method. As a result, the thickness of the film was found to be 40 angstroms. This sample was called an it/st-PMMA/COOH surface block.

### Example A-2

### it/st-PMMA/COOH Surface Chip

(1) Preparation of Isotactic-Polymethyl Methacrylate Solution (0.3% it-PMMA)

0.3 g of isotactic-polymethyl methacrylate (number average molecular weight: 23,000; hereinafter referred to as it-PMMA) was dissolved in 100 ml of acetonitrile to prepare 0.3% it-PMMA.

(2) Preparation of Syndiotactic-Polymethyl Methacrylate Solution (0.3% st-PMMA)

0.3 g of syndiotactic-polymethyl methacrylate (number average molecular weight: 23,000; hereinafter referred to as st-PMMA) was dissolved in 100 ml of acetonitrite to prepare 0.3% st-PMMA.

(3) Production of it/st-PMMA/COOH Surface Chip

Gold was evaporated onto a cover glass with a square of 1 cm, such that the thickness of a metal film became 500 angstroms, so as to obtain a gold chip. This gold chip was treated with a Model-208 UV-ozone cleaning system (TECHNOVISION INC.) for 30 minutes, and it was then immersed in 100 ml of 0.3% it-PMMA for 15 minutes. Subsequently, this gold chip was immersed in 50 ml of acetonitrile 5 times each for 1 minute. After the chip had been immersed in acetonitrile 5 times, acetonitrile attached to the surface of the gold chip was removed by nitrogen blowing.

Subsequently, this gold chip was immersed in 100 ml of 0.3% st-PMMA for 15 minutes. Thereafter, it was immersed in 50 ml of acetonitrile 5 times each for 1 minute. After the gold chip had been immersed in acetonitrile 5 times, acetonitrile attached to the surface of the gold chip was removed by nitrogen blowing. When the thickness of an it/st-PMMA film was measured by the ellipsometry method, the thickness of the film was found to be 50 angstroms. This sample was called an it/st-PMMA surface chip.

The it/st-PMMA surface chip was immersed in an NaOH aqueous solution (1 N) at 40° C. for 16 hours. Thereafter, it was washed with water 3 times, and the water was then removed by nitrogen blowing. When the thickness of an it/st-PMMA/COOH film was measured by the ellipsometry method, the thickness of the film was found to be 50 angstroms. This sample was called an it/st-PMMA/COOH surface chip.

### Comparative Example A-1

### it/st-PMMA Alternatively Laminated Block

The it/st-PMMA alternatively laminated block produced by the method described in Example 1 was defined as Comparative example A-1.

### Comparative Example A-2

### it/st-PMMA Block

(1) Preparation of Isotactic Polymethyl Methacrylate Solution (1.0% it-PMMA)

1.0 g of it-PMMA was dissolved in 100 ml of acetonitrile to prepare 1.0% it-PMMA.

(2) Production of it-PMMA Surface Block

A gold block was treated with a Model-208 UV-ozone cleaning system (TECHNOVISION INC.) for 30 minutes. Thereafter, 1.0% it-PMMA was added dropwise to the surface coated with gold via evaporation, and it was then left at rest for 15 minutes. Subsequently, the above block was immersed in 50 ml of acetonitrile 5 times each for 1 minute, so that 1.0% it-PMMA attached to the surface coated with gold via evaporation was substituted with acetonitrile. After completion of the substitution, acetonitrile attached to the surface of the block was removed by nitrogen blowing. When the thickness of an it-PMMA film was measured by the ellipsometry method, the thickness of the film was found to be 40 angstroms. This sample was called an it-PMMA surface block.

### Comparative Example A-3

### st-PMMA Block

An st-PMMA surface block was produced by the same method as in Comparative example 2 with the exception that st-PMMA was used instead of it-PMMA. The thickness of the st-PMMA film was found to be 20 angstroms.

### Comparative Example A-4

### it-PMMA/COOH Surface Block

The same operations as in Example A-1 were performed on the block of Comparative example A-2, so as to produce an it-PMMA/COOH surface block. The thickness of the it-PMMA/COOH film was found to be 40 angstroms.

### Comparative Example A-5

### st-PMMA/COOH Surface Block

The same operations as in Example A-1 were performed on the block of Comparative example A-3, so as to produce an st-PMMA/COOH surface block. The thickness of the st-PMMA/COOH film was found to be 20 angstroms.

### Comparative Example A-6

### SAM Surface Block

A gold block having a thickness of a film coated with gold via evaporation of 50 nm was treated with an ozone cleaner for 30 minutes. Thereafter, the block was immersed in an ethanol solution containing 1 mM 7-carboxy-1-heptanethiol for 18 hours, so as to carry out a surface treatment. Thereafter, it was washed with ethanol 5 times, with a mixed solvent consisting of ethanol and water 1 time, and then with water 5 times. By these operations, an SAM surface block coated with an SAM compound (7-carboxy-1-heptanethiol) was obtained.

### Comparative Example A-7

### Gold Block

A gold block produced by the method described in Example A-1 was defined as Comparative example A-7.

Evaluation 1: Nonspecific Adsorption of Proteins

Nonspecific adsorption of proteins on the surface of a biosensor becomes a cause of noise. Accordingly, it is preferable that such nonspecific adsorption of proteins could occur to an extremely small extent. Nonspecific adsorption of BSA (manufactured by Sigma) and avidin (manufactured by Nacalai Tesque) was measured.

Bach of the products produced in Examples A-1 and A-2 and Comparative examples A-1 to A-7 was placed in the device shown in FIG. 22 of Japanese Patent Laid-Open (Kokai) No. 2001-330560 (hereinafter referred to as the surface plasmon resonance measurement device of the present invention), and it was then blocked with ethanolamine, followed by measurement. The blocking treatment with ethanolamine was carried out by adding dropwise to the sensor surface of the block a mixed solution consisting of 1-ethyl-2,3-dimethylaminopropylcarbodiimide (400 mM) and N-hydroxysuccinimide (100 mM), and leaving at rest for 60 minutes. Then, the resultant product was washed with water. Thereafter, an ethanolamine-HCl solution (1 M, pH 8.5) was added to each measurement block, and it was left at rest for 20 minutes. Thereafter, it was washed with an HBS-EP buffer (manufactured by Biacore; pH 7.4). It is to be noted that the composition of the above used HBS-EP buffer consisted of 0.01 mol/l HEPES (N-2-hydroxyethylpiperazin-N′-2-ethanesulfonic acid) (pH 7.4), 0.15 mol/l NaCl, 0.003 mol/l EDTA, and 0.005%-by-weight Surfactant P20. Thereafter, a BSA solution (2 mg/ml, HBS-EP buffer) or avidin solution (2 mg/ml, HBS-EP buffer) was added thereto, followed by leaving at rest for 10 minutes. Thereafter, the resultant product was washed with an HBS-EP buffer, and 3 minutes later, the amount of a change in resonance signals was measured. The change amount was evaluated from a relative value with respect to the change amount of the gold block (Comparative example A-7). The evaluation results are shown in Table 1.

TABLE 1

| | | Nonspecific | |
|---|---|---|---|
| | | adsorption | |
| | | of proteins | |
| Sample | | BSA | Avidin |
| Example A-1 | it/st-PMMA/COOH surface block | 0.1 | 0.2 |
| Example A-2 | it/st-PMMA/COOH surface chip | 0.1 | 0.2 |
| Comparative | it/st-PMMA alternatively laminated block | 0.2 | 0.3 |
| example A-1 | | | |
| Comparative | it-PMMA block | 0.4 | 0.6 |
| example A-2 | | | |
| Comparative | st-PMMA block | 0.5 | 0.8 |
| example A-3 | | | |
| Comparative | it-PMMA/COOH surface block | 0.4 | 0.7 |
| example A-4 | | | |
| Comparative | st-PMMA/COOH surface block | 0.5 | 0.8 |
| example A-5 | | | |
| Comparative | SAM surface block | 0.4 | 0.7 |
| example A-6 | | | |

Evaluation 2: Measurement of Interaction Between Protein and Test Compound

Neutral avidin (manufactured by PIERCE; hereinafter referred to as N-avidin) was immobilized on each of the measurement blocks produced in Example A-1 and Comparative examples A-1 and A-6, and the interaction between the protein and D-biotin (manufactured by Nacalai Tesque) was measured by the method described below.

A mixed solution consisting of 1-ethyl-2,3-dimethylaminopropylcarbodiimide (400 mM) and N-hydroxysuccinimide (100 mM) was added to the measurement block, followed by leaving at rest for 20 minutes. Thereafter, the resultant block was washed with an HBS-N buffer (manufactured by Biacore; pH 7.4). Subsequently, an N-avidin solution (100 μg/ml; HBS-N buffer) was added thereto, followed by leaving at rest for 30 minutes. Thereafter, the resultant block was washed with an HBS-N buffer. By these operations, N-avidin was immobilized on the surface of each measurement chip by covalent bonding. The amount by which resonance signals obtained before the addition of N-avidin and after the washing of N-avidin had changed was defied as the immobilized amount of N-avidin. N-avidin was immobilized on the it/st-PMMA/COOH surface block of the present invention, as in the case of the SAM surface block. It is to be noted that the composition of the above used HBS-N buffer consisted of 0.01 mol/l HEPES (N-2-hydroxyethylpiperazin-N′-2-ethanesulfonic acid) (pH 7.4) and 0.15 mol/l NaCl.

Furthermore, an ethanolamine-HCl solution (1 M, pH 8.5) was added to the measurement block, and then washed with an HBS-N buffer, so that COOH groups remaining without reacting with N-avidin were blocked.

Subsequently, the measurement block was placed in the surface plasmon resonance measurement device of the present invention, and D-biotin (0.5 μg/ml, HBS-N buffer) was added to the measurement block, followed by leaving at rest for 10 minutes. Thereafter, it was washed with an HBS-N buffer. The amount by which resonance signals obtained before the addition of D-biotin and after the washing of D-biotin had changed was defined as the binding amount of D-biotin to N-avidin. As in the case of the SAM surface block, D-biotin was detected from the it/st-PMMA/COOH surface block of the present invention. The immobilized amount of N-avidin and the detected amount of D-biotin were evaluated from relative values with respect to those of the SAM surface block (Comparative example A-6). The evaluation results are shown in Table 2.

TABLE 2

| | | Immobilized | Detected |
|---|---|---|---|
| | | amount of | amount of |
| Sample | | N-avidin | D-biotin |
| Example A-1 | it/st-PMMA/COOH surface | 1 | 1 |
| | block | | |
| Comparative | it/st-PMMA alternatively | 0 | 0 |
| example A-1 | laminated block | | |
| Comparative | SAM surface block | 1 | 1 |
| example A-6 | | | |

As is clear from the above results, when the solid substrate used for sensors of the present invention is used, nonspecific adsorption of proteins occurred to an extremely small extent, and thus, immobilization of a protein and detection of a test compound could be carried out by surface plasmon resonance. In addition, each measurement block was immersed in a fluorescent-labeled substrate FITC-avidin solution (1 mg/ml, HBS-EP buffer) for 15 minutes, and it was then washed with water and then observed with a fluorescence microscope. A fluorescence derived from FITC was observed in the sample of comparative examples. In contrast, no fluorescence was observed in the sample of the present invention. As a result, it was found that the solid substrate used for sensors of the present invention has a surface that causes only an extremely small degree of nonspecific adsorption.

### Example B-1

### PMMA/PSt Block (1)

(1) Preparation of Polymethyl Methacrylate-Polystyrene Copolymer Solution (0.1% PMMA/PSt (1))

0.1 g of a polymethyl methacrylate-polystyrene copolymer (number average molecular weight: 60,000; polymethyl methacrylate: polystyrene=1:1 (weight ratio)) was dissolved in a mixed solution consisting of 60 ml of 2-butanone and 40 ml of ethanol, so as to prepare 0.1% PMMA/PSt (1).

The lower limited liquid temperature of this solution, at which no polymer deposits are generated, was 18° C.

(2) Production of Gold Block

Gold was evaporated onto the dielectric block shown in FIG. 23 of Japanese Patent Laid-Open (Kokai) No. 2001-330560, such that the thickness of a gold film became 500 angstroms, so as to obtain a gold block.

(3) Production of PMMA/PSt Block (1)

The gold block was treated with a Model-208 UV-ozone cleaning system (TECHNOVISION INC.) for 30 minutes. Thereafter, 0.1% PMMA/PSt (1) was added dropwise to the surface coated with gold via evaporation, and it was then left at rest for 15 minutes. Subsequently, the above block was immersed in a mixed solution consisting of 30 ml of 2-butanone and 20 ml of ethanol at 25° C. 5 times each for 1 minute, so that 0.1% PMMA/PSt attached to the surface coated with gold via evaporation was substituted with the mixed solution consisting of 30 ml of 2-butanone and 20 ml of ethanol. After completion of the substitution, the mixed solution attached to the surface of the block was removed by nitrogen blowing, followed by drying in a vacuum for 16 hours. The thickness of a PMMA/PSt film was measured by the ellipsometry method (In-Situ Ellipsometer MAUS-101; manufactured by Five Lab). As a result, the thickness of the film was found to be 50 angstroms. This sample was called PMMA/PSt block (1).

### Example B-2

### PMMA/PSt Block (2)

(1) Preparation of Polymethyl Methacrylate-Polystyrene Copolymer Solution (0.1% PMMA/PSt (2))

0.1 g of a polymethyl methacrylate-polystyrene copolymer (number average molecular weight: 60,000, polymethyl methacrylate: polystyrene=1:1 (weight ratio)) was dissolved in a mixed solution consisting of 45 ml of 2-butanone and 55 ml of acetonitrile, so as to prepare 0.1% PMMA/PSt (2).

The lower limited liquid temperature of this solution, at which no polymer deposits are generated, was 20° C.

(2) Production of PMMA/PSt Block (2)

A sample was produced by the same operations as in Example B-1(3), with the exception that 0.1% PMMA/Pst(2) was used instead of 0.1% PMMA/Pst(1), and a mixed solution consisting of 45 ml of 2-butanone and 55 ml of acetonitrile was used instead of a mixed solution consisting of 30 ml of 2-butanone and 20 ml of ethanol. When the thickness of a PMMA/PSt film was measured by the ellipsometry method (In-Situ Ellipsometer MAUS-101; manufactured by Five Lab), the thickness of the film was found to be 50 angstroms. This sample was called PMMA/PSt block (2).

### Example B-3

### PMMA/PSt Block (3)

A sample was produced by the same operations as in Example B-1(3) with the exception that the liquid temperature of a mixed solution was set at 60° C. The thickness of a PMMA/PSt film was found to be 30 angstroms. This sample was called PMMA/PSt block (3).

### Example B-4

### PMMA PSt/COOH Block (1)

PMMA/PSt block (1) was immersed in an NaOH aqueous solution (1 N) at 40° C. for 16 hours. Thereafter, the block was washed with water 3 times, and the water was then removed by nitrogen blowing. As a result of measurement by the ellipsometry method, the thickness of a PMMA/PSt/COOH film was found to be 50 angstroms. This sample was called PMMA/PSt/COOH block (1).

### Example B-5

### PMMA/PSt COOH Block (2)

The same operations as in Example B-4 were performed on the PMMA/PSt block (2), so as to obtain PMMA/PSt/COOH block (2). As a result of measurement by the ellipsometry method, the thickness of a PMMA/PSt/COOH film was found to be 50 angstroms.

### Example B-6

### PMMA/PSt/COOH Block (3)

The same operations as in Example B-4 were performed on the PMMA/PSt block (3), so as to obtain PMMA/PSt/COOH block (3). As a result of measurement by the ellipsometry method, the thickness of a PMMA/PSt/COOH film was found to be 10 angstroms.

### Comparative Example B-1

### PMMA/PSt Block (4)

(1) Preparation of Polymethyl Methacrylate-Polystyrene Copolymer Solution (0.1% PMMA/PSt (4))

0.1 g of a polymethyl methacrylate-polystyrene copolymer (number average molecular weight: 60,000; polymethyl methacrylate: polystyrene=1:1 (weight ratio)) was dissolved in 100 ml of 2-phenoxyethanol, so as to prepare 0.1% PMMA/PSt (4).

(2) Production of PMMA/PSt Block

A gold block was treated with a Model-208 UV-ozone cleaning system (TECHNOVISION INC.) for 30 minutes. Thereafter, 0.1% PMMA/PSt was added dropwise to the surface coated with gold via evaporation, and it was then left at rest for 15 minutes. Subsequently, the above block was immersed in 50 ml of 2-phenoxyethanol 5 times each for 1 minute, so that 0.1% PMMA/PSt attached to the surface coated with gold via evaporation was substituted with 2-phenoxyethanol. Moreover, the block was immersed in 50 ml of ethanol 5 times each for 1 minute, so that 2-phenoxyethanol attached to the surface coated with gold via evaporation was substituted with ethanol. After completion of the substitution, ethanol attached to the surface of the block was removed by nitrogen blowing, followed by drying in a vacuum for 16 hours. As a result of measurement by the ellipsometry method (In-Situ Ellipsometer MAUS-101; manufactured by Five Lab), the thickness of a PMMA/PSt film was found to be 10 angstroms. This sample was called PMMA/PSt block (4).

### Comparative Example B-2

### PMMA/PSt/COOH Block (4)

A sample was produced from the PMMA/PSt block (4) by the same operations as in Example B-4. The thickness of a PMMA/PSt/COOH film was found to be 10 angstroms. This sample was called PMMA/PSt/COOH block (4).

### Comparative Example B-3

### SAM Block

A gold block having a thickness of a film coated with gold via evaporation of 50 nm was treated with an ozone cleaner for 30 minutes. Thereafter, the block was immersed in an ethanol solution containing 1 mM 7-carboxy-1-heptanethiol for 18 hours, so as to carry out a surface treatment. Thereafter, it was washed with ethanol 5 times, with a mixed solvent consisting of ethanol and water 1 time, and then with water 5 times. By these operations, an SAM block coated with an SAM compound (7-carboxy-1-heptanethiol) was obtained.

### Comparative Example B-4

### Gold Block

A gold block produced by the method described in Example B-1 was defined as Comparative example B-4.

Evaluation 1: Nonspecific Adsorption of Proteins

Nonspecific adsorption of proteins on the surface of a biosensor becomes a cause of noise. Accordingly, it is preferable that such nonspecific adsorption of proteins could occur to an extremely small extent. Nonspecific adsorption of BSA (manufactured by Sigma) and avidin (manufactured by Nacalai Tesque) was measured.

Ethanol Blocking Treatment

Each of the PMMA/PSt/COOH block and the SAM block was placed in the device shown in FIG. 22 of Japanese Patent Laid-Open (Kokai) No. 2001-330560 (hereinafter referred to as the surface plasmon resonance measurement device of the present invention), and it was then blocked with ethanolamine, followed by measurement. The blocking treatment with ethanolamine was carried out by adding dropwise to the sensor surface of the block a mixed solution consisting of 1-ethyl-2,3-dimethylaminopropylcarbodiimide (400 mM) and N-hydroxysuccinimide (100 mM), and leaving at rest for 60 minutes. Then, the resultant block was washed with water. Thereafter, an ethanolamine-HCl solution (1 M, pH 8.5) was added to each measurement block, and it was left at rest for 20 minutes. Thereafter, it was washed with an HBS-EP buffer (manufactured by Biacore; pH 7.4). It is to be noted that the composition of the above used HBS-EP buffer consisted of 0.01 mol/l HEPES (N-2-hydroxyethylpiperazin-N′-2-ethanesulfonic acid) (pH 7.4), 0.15 mol/l NaCl, 0.003 mol/l EDTA, and 0.005%-by-weight Surfactant P20.

Measurement of Nonspecific Adsorption

Each of PMMA/PSt blocks and other blocks blocked with ethanolamine was placed in the surface plasmon resonance measurement device of the present invention, and it was then washed with an HBS-EP buffer. Thereafter, a BSA solution (2 mg/ml, HBS-EP buffer) or avidin solution (2 mg/ml, HBS-EP buffer) was added thereto, followed by leaving at rest for 10 minutes. Thereafter, the resultant block was washed with an HBS-EP buffer, and 3 minutes later, the amount of a change in resonance signals was measured. The change amount was evaluated from a relative value with respect to the change amount of the gold block (Comparative example B-4).

TABLE 3

| | | Nonspecific | |
|---|---|---|---|
| | | adsorption | |
| | | of proteins | |
| Sample | | BSA | Avidin |
| Example B-1 | PMMA/PSt surface block (1) | 0.1 | 0.2 |
| Example B-2 | PMMA/PSt surface block (2) | 0.1 | 0.2 |
| Example B-3 | PMMA/PSt surface block (3) | 0.2 | 0.3 |
| Example B-4 | PMMA/PSt/COOH surface block (1) | 0.1 | 0.2 |
| Example B-5 | PMMA/PSt/COOH surface block (2) | 0.1 | 0.2 |
| Example B-6 | PMMA/PSt/COOH surface block (3) | 0.2 | 0.3 |
| Comparative | PMMA/PSt surface block (4) | 0.4 | 0.5 |
| example B-1 | | | |
| Comparative | PMMA/PSt/COOH surface block (4) | 0.4 | 0.6 |
| example B-2 | | | |
| Comparative | SAM surface block | 0.4 | 0.7 |
| example B-3 | | | |

Evaluation 2: Measurement of Interaction Between Protein and Test Compound

Neutral avidin (manufactured by PIERCE; hereinafter referred to as N-avidin) was immobilized on each of the measurement blocks produced in Examples B-4 to B-6 and Comparative examples B-2 and B-3, and the interaction between the protein and D-biotin (manufactured by Nacalai Tesque) was measured by the method described below.

A mixed solution consisting of 1-ethyl-2,3-dimethylaminopropylcarbodiimide (400 mM) and N-hydroxysuccinimide (100 mM) was added to the measurement block, followed by leaving at rest for 20 minutes. Thereafter, the resultant block was washed with an HBS-N buffer (manufactured by Biacore; pH 7.4). Subsequently, an N-avidin solution (100 μg/ml; HBS-N buffer) was added thereto, followed by leaving at rest for 30 minutes. Thereafter, the resultant block was washed with an HBS-N buffer. By these operations, N-avidin was immobilized on the surface of each measurement chip by covalent bonding. The amount by which resonance signals obtained before the addition of N-avidin and after the washing of N-avidin had changed was defined as the immobilized amount of N-avidin. The immobilized amount was evaluated from a relative value with respect to the change amount of the SAM block (Comparative example B-3). The evaluation results are shown in Table 4. Larger the relative value of the change amount, larger the immobilized amount that can be obtained. Thus, it is preferable that the relative value be large. It is to be noted that the composition of the above used HBS-N buffer consisted of 0.01 mol/l HEPES (N-2-hydroxyethylpiperazin-N′-2-ethanesulfonic acid) (pH 7.4) and 0.15 mol/l NaCl.

Furthermore, an ethanolamine-HCl solution (1 M, pH 8.5) was added to the measurement block, and then washed with an HBS-N buffer, so that COOH groups remaining without reacting with N-avidin were blocked.

Subsequently, the measurement block was placed in the surface plasmon resonance measurement device of the present invention, and D-biotin (0.5 μg/ml, HBS-N buffer) was added to the measurement block, followed by leaving at rest for 10 minutes. Thereafter, it was washed with an HBS-N buffer. The amount by which resonance signals obtained before the addition of D-biotin and after the washing of D-biotin had changed was defined as the binding amount of D-biotin to N-avidin. The binding amount was evaluated from a relative value with respect to the change amount in the SAM block (Comparative example B-3). The evaluation results are shown in Table 4. Larger the relative value of the change amount, higher the detection sensitivity that can be obtained. Thus, it is preferable that the relative value be large.

TABLE 4

| | | Immobilized | Detected |
|---|---|---|---|
| | | amount of | amount of |
| Sample | | N-avidin | D-biotin |
| Example B-4 | PMMA/PSt/COOH surface | 1 | 1 |
| | block (1) | | |
| Example B-5 | PMMA/PSt/COOH surface | 1 | 1 |
| | block (2) | | |
| Example B-6 | PMMA/PSt/COOH surface | 1 | 1 |
| | block (3) | | |
| Comparative | PMMA/PSt/COOH surface | 0 | 0 |
| example B-2 | block (4) | | |

From the results shown in Table 3, it was found that the surface formation method of the present invention provides a surface plasmon resonance substrate causing an extremely small degree of nonspecific adsorption of proteins. The surface of each sample was immersed in a fluorescent-labeled substrate FITC-avidin solution (1 mg/ml, HBS-EP buffer) for 15 minutes, and it was then washed with water and then observed with a fluorescence microscope. A fluorescence derived from FITC was observed in the SAM block. In contrast, no fluorescence was observed in the sample of the present invention. As a result, it was found that the surface formation method of the present invention provides a surface that causes only an extremely small degree of nonspecific adsorption.

From the results shown in Table 4, it was found that a sensor substrate produced by the surface formation method of the present invention enables immobilization of a protein and detection of a test compound.

In addition, in the case of the measurement block of Comparative example B-1 produced with a single solvent, in order to form a surface suppressing nonspecific adsorption, approximately 50 types of solvents require to be evaluated in terms of solubility of polymers and in terms of the nonspecific adsorption of the produced measurement block. Thus, an enormous amount of work has been required for the development of the above measurement block. In contrast, the measurement block of the present invention has been developed by mixing any given good solvents and poor solvents for polymers, thereby significantly reducing the time and work necessary for the development.

### EFFECT OF THE INVENTION

The solid substrate used for sensors of the present invention enables suppression of nonspecific adsorption and detection of a substance interacting with a specific physiologically active substance. The method of the present invention for producing a solid substrate enables adsorption of various hydrophobic polymers on the surface of the solid substrate, thereby providing a solid substrate used for sensors that suppresses nonspecific adsorption. Moreover, it also becomes possible to provide a solid substrate used for sensors that suppresses nonspecific adsorption, regardless of whether or not the solid substrate has a planar form.

1. A solid substrate used for sensors, wherein two or more different hydrophobic polymer layers are laminated on the solid substrate, and among the above hydrophobic polymer layers, the surface of a layer, which is farthest from the solid substrate, is modified.

2. The solid substrate used for sensors according to claim 1, wherein the surface-modified hydrophobic polymer layer has a functional group capable of generating a covalent bond.

3. The solid substrate used for sensors according to claim 1, wherein the solid substrate has one or more holes or projections on the surface thereof, and the projected area of the aforementioned hole or projection observed from the top of the substrate is between 0.001 mm² and 10,000 mm², and the depth or height of the aforementioned hole or projection is between 100 nm and 10 cm.

4. The solid substrate used for sensors according to claim 1, wherein a metal film exists between the solid substrate and the hydrophobic polymer layer.

5. The solid substrate used for sensors according to claim 1, wherein the metal film consists of a free electron metal selected from the group consisting of gold, silver, copper, platinum, and aluminum.

6. The solid substrate used for sensors according to claim 1, wherein the surface modified hydrophobic polymer layer has a functional group capable of immobilizing a physiologically active substance.

7. The solid substrate used for sensors according to claim 1, wherein the functional group capable of immobilizing a physiologically active substance is —OH, —SH, —COOH, —NR¹R² (wherein R¹ and R² each independently represents a hydrogen atom or lower alkyl group), —CHO, —NR³NR¹R² (wherein each of R¹, R², and R³ independently represents a hydrogen atom or lower alkyl group), —NCO, —NCS, an epoxy group, or a vinyl group.

8. The solid substrate used for sensors according to claim 1, which is used in non-electrochemical detection.

9. The solid substrate used for sensors according to claim 1, which is used in surface plasmon resonance analysis.

10. A method for producing the solid substrate used for sensors according to claim 1 which comprises steps of allowing two or more types of hydrophobic polymer solutions to come into contact with a solid substrate in turns, and modifying the surface of the obtained solid substrate.

11. A method for producing a solid substrate used for sensors, to the surface of which a physiologically active substance binds; wherein the method comprises a step of allowing the physiologically active substance to come into contact with the surface of the solid substrate used for sensors according to claim 1, so as to immobilize the substance thereon.

12. The solid substrate used for sensors according to claim 1, to the surface of which a physiologically active substance binds.

13. A method for detecting or measuring a substance interacting with a physiologically active substance, which comprises steps of allowing the physiologically active substance to come into contact with the surface of the solid substrate used for sensors according to claim 1, so as to immobilize the substance thereon, and allowing the obtained solid substrate used for sensors, to the surface of which the physiologically active substance binds, to come into contact with a test substance.

14. A method for producing a solid substrate used for sensors which comprises steps of allowing a solid substrate to come into contact with a hydrophobic polymer solution and then allowing it come into contact with a mixed solution comprising two or more organic solvents, which does not contain the above polymer.

15. The method according to claim 14 which further comprises a step of modifying the surface of the obtained solid substrate.

16. The method according to claim 14 wherein the mixed solution comprising two or more organic solvents, which does not contain the polymer, comprises a good solvent and a poor solvent for the polymer.

17. The method according to claim 14 wherein the mixed solution comprising two or more organic solvents, which does not contain the above polymer, is used at a liquid temperature that is 1° C. to 50° C. higher than the lower limit liquid temperature at which no hydrophobic polymer deposits are generated when the concentration of the above mixed solution is adjusted to the same concentration as that of the above hydrophobic polymer solution containing hydrophobic polymers.

18. The method according to claim 14 wherein the solvent contained in the hydrophobic polymer solution is identical to the solvent contained in the solution, which does not contain the polymer.

19. The method according to claim 14 wherein the surface modification involves introduction of a functional group capable of generating a covalent bond.

20. The method according to claim 14 wherein the solid substrate, which is allowed to come into contact with the hydrophobic polymer solution, has a metal surface or is coated with a metal film.

21. The method according to claim 14 wherein the solid substrate used for sensors is used in surface plasmon resonance analysis.

22. A method for producing a solid substrate used for sensors, to the surface of which a physiologically active substance binds, wherein the above method comprises steps of producing a solid substrate used for sensors by the method of claim 14, and allowing a physiologically active substance to come into contact with the surface of the obtained solid substrate used for sensors, so as to immobilize the substance thereon.

23. A method for detecting or measuring a substance interacting with a physiologically active substance, wherein the above method comprises steps of producing a solid substrate used for sensors by the method of claim 14, allowing the physiologically active substance to come into contact with the surface of the obtained solid substrate used for sensors, so as to immobilize the substance thereon, and allowing the obtained solid substrate used for sensors, to the surface of which the physiologically active substance binds, to come into contact with a test substance.
